(19) United States
(12) Patent Application Publication
(10) Pub. No.: US 2025/0000658 A1
(43) Pub. Date: Jan. 2, 2025

(54) ACETABULAR DEVICE AND MODULAR NECK ASSEMBLY

(71) Applicant: ALM Ortho, Inc., Woolwich, ME (US)

(72) Inventors: David Steven Feldman, Lantana, FL (US); Donald Steven Block, Warsaw, IN (US); Brian R. McLaughlin, Yarmouth, ME (US)

(21) Appl. No.: 18/755,969

(22) Filed: Jun. 27, 2024

Related U.S. Application Data
(60) Provisional application No. 63/523,695, filed on Jun. 28, 2023.

(51) Int. Cl. A61F 2/34; A61F 2/30

(52) U.S. Cl. CPC A61F 2/34; A61F 2002/30354; A61F 2002/30433; A61F 2002/30785; A61F 2002/30884; A61F 2002/3482

(57) ABSTRACT

A multi-piece acetabular device and modular neck segment for temporary implantation during a distraction procedure.

### BACKGROUND INFORMATION

### Field of the Invention

The invention relates to devices used in distraction osteogenesis and histiogenesis, and in particular, devices used in distraction procedures for orthopedic surgery. High dislocation of the hip for various etiologies is a difficult problem to correct when a hip replacement is required to alleviate pain and improve function.

### Discussion of Prior Art

Distraction histiogenesis is a process used in orthopedic surgery, podiatric surgery, and oral and maxillofacial surgery to alleviate skeletal issues through reconstructive surgery. Generally, the procedure involves cutting and separating a bone in order to slowly expand the bone over time. It can also be used to slowly stretch soft tissues including muscles, ligaments and nerves in order to safely lengthen various locations in the body.

The process is typically achieved over a number of phases. If bone is to be lengthened, during phase one the bone is cut and a device fitted or mounted to each side of the cut for use in the next phase. The second phase is a latency period during which early stages of bone healing are allowed. The third phase is the “distraction phase”, where the device mounted to the bone is used to slowly separate the two pieces to allow new bone to grow and fill in the gap that is created between the two sections of bone. Once the desired length is achieved, usually over a number of days or weeks, the fourth phase, known as the consolidation phase, commences during which the device keeps the bone in a stable position as it fully heals. Finally, the last phase of the process is to remove the device.

Another important use of distraction histogenesis is the slow distraction of soft tissues, such as muscles and nerves. This occurs concomitantly with bone lengthening but also can be utilized as a stand-alone procedure.

One of the needs in orthopedics of distraction histiogenesis is in the treatment of a chronic high dislocation of the hip. The etiology of this can be congenital, post-traumatic, post-infectious or can be related to a skeletal dysplasia such as Larsen Syndrome and Diastrophic Dysplasia. Historically, these chronic dislocations were treated with total hip replacements that required extensive soft tissue releases and femoral shortening of as much as 5 centimeters. The muscle release would often cause permanent weakness and a limp and the tension of acutely pulling the femur down would at times be complicated by a peroneal and or sciatic nerve palsy.

Slowly distalizing the femur, with distraction histiogenesis, is a methodology that can avoid these complications.

What is needed, therefore, is a device that facilitates an easier surgical technique and assembly of the distraction components.

### BRIEF SUMMARY OF THE INVENTION

The invention is a custom-designed, 2-piece acetabular device and modular neck segment for temporary implantation during a distraction procedure. For example, it is particularly well suited for a hip distraction procedure to reposition the femur and allow for total hip arthroplasty (“THA”) to address chronic hip dislocation.

The device is constructed to facilitate an easier surgical technique and assembly of the distraction components. The acetabular device is comprised of a two-piece shell, with one of the two pieces fitted directly to the bone and designed for a precise fit with the bone surface for initial stability. Compression bone screws are used to initially fix the first shell to the bone. A conventional femoral head is secured to the top of the modular neck segment and topped with a custom liner. The femoral head, capped with the liner, is then placed against the first half of the shell, after which the second half is placed in position and secured with additional compression screws.

Once the procedure is complete, the acetabular device and modular neck is left in place for a number of weeks, typically six to eight, after which the device is removed, and a total hip arthroplasty is performed.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1 is a design drawing showing the device fully assembled against the bone.

FIG. 2 is a design drawing showing the device assembled against the bone without the lateral shell.

FIG. 3 is a design drawing of the medial shell.

FIG. 4 is a design drawing of the liner.

FIG. 5 is a design drawing of the lateral shell.

FIG. 6 is a design drawing of the machine screw.

FIG. 7 is a design drawing of the medial shell placed against the bone.

FIG. 8 is a design drawing of the medial shell placed against the bone with the first two screws inserted into the bone.

FIG. 9 is a front view of the neck separated from the OEM IM Nail.

FIG. 10 is a front view of the neck inserted into a cross-sectional view of the nail.

FIG. 11 is a front view of the neck inserted into a cross-sectional view of the nail with the femoral head on top of the neck.

FIG. 12 illustrates the device performing its function.

FIG. 13 illustrates the device attached to the hipbone and the leg, without the lateral section.

FIG. 14 illustrates the lateral section being placed over the liner against the medial shell.

FIG. 15 illustrates the lateral section in the operational position.

FIG. 16 illustrates the machine screw securing the lateral shell to the medial shell.

FIG. 17 illustrates the medial shell.

FIG. 18 illustrates the inner side of the medial shell.

FIG. 19 illustrates the inner side of the lateral shell.

FIG. 20 illustrates the machine screw.

FIG. 21 illustrates a first embodiment of the neck segment.

FIG. 22 illustrates a second embodiment of the neck segment.

FIG. 23 is a side view of the liner.

FIG. 24 is a perspective view of the liner.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

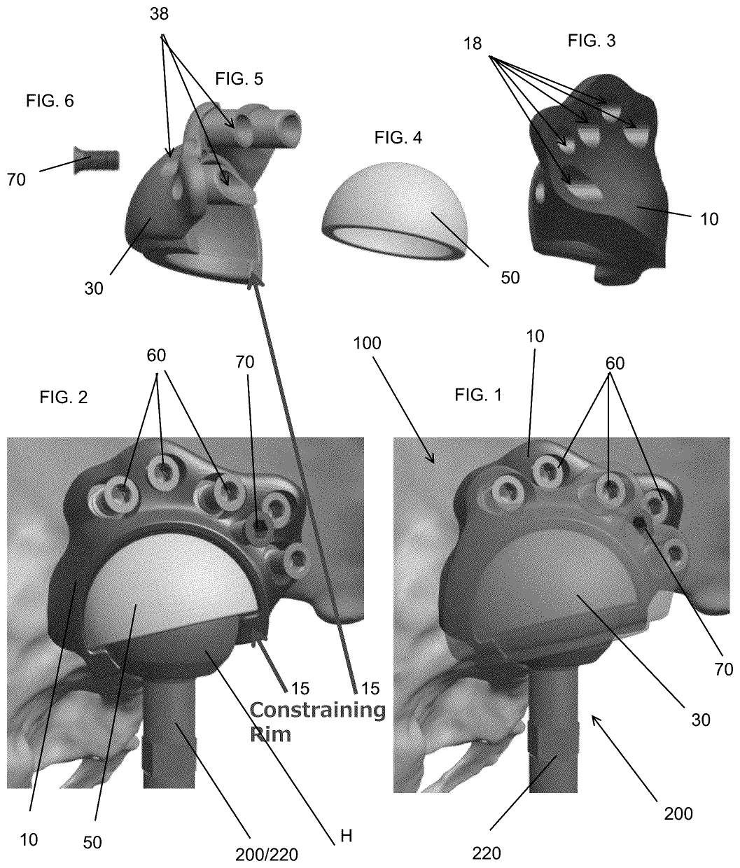

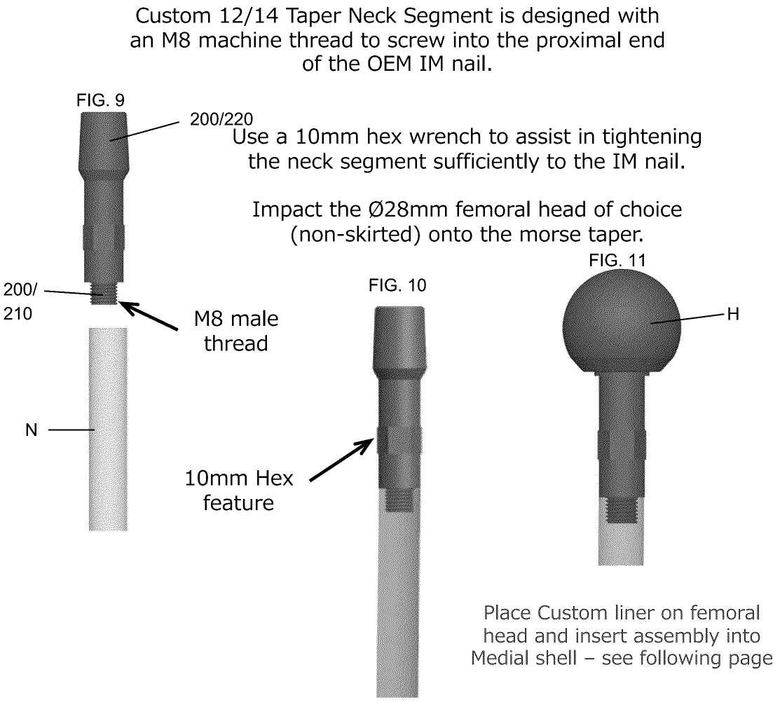

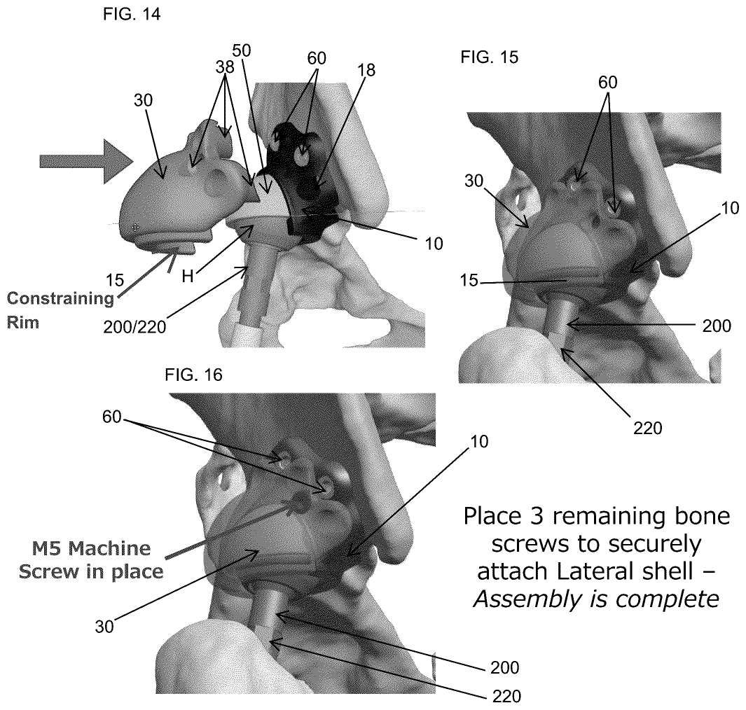

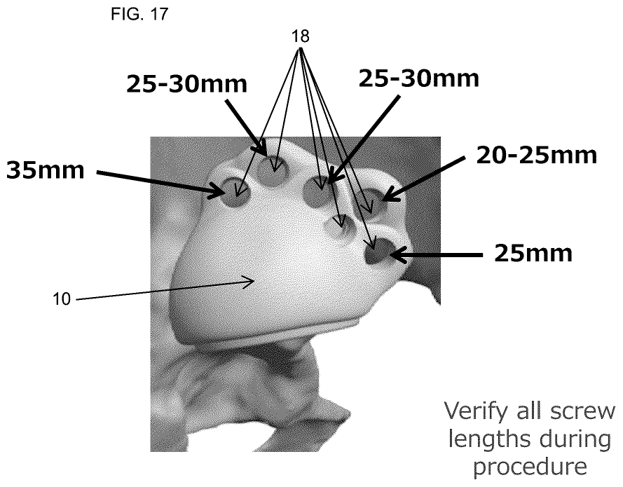

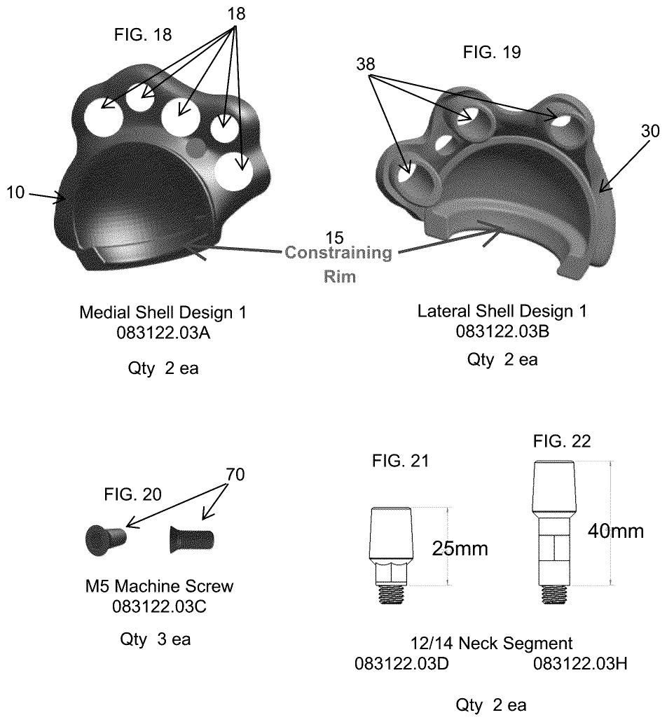

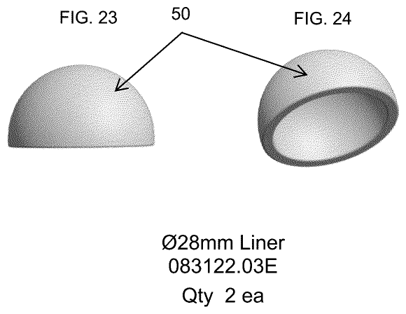

FIGS. 1-24 illustrate an acetabular device 100 with a modular neck 200 for temporary implantation in a distraction procedure. The acetabular device 100 includes a medial shell 10 that is adapted to fit against a bone structure, a lateral shell 30 that secures to the medial shell, and a liner 50 that fits inside the two shells 10, 30, with the medial shell 10 and lateral shell 30 each having a constraining rim 15 that ultimately secures a femoral head prosthesis H.

The modular neck 200 has a first end 210 that is a threaded taper neck segment that is sized to screw into the proximal end of a conventional IM hip nail N. The modular neck 200 has a second end 220 that is adapted to attach to a conventional femoral head prosthesis H, the prosthesis H having an opening and the second end 220 having a shape that causes the components to secure to one another by a press fit or interference fit. The femoral head H is inserted into the liner 50 and secured against the medial shell 10 and the lateral shell 30 by the constraining rim 15.

More specifically, the medial shell 10 has an outer side 12 that is shaped to fit directly to the anatomical acetabulum in the ilium in the region of the anterior gluteal line. Each medial shell 10 is customized to fit closely with the bone surface of a given patient for initial stability. The medial shell has an inner side 14 that is semi-circular in shape so as to support the liner 50 into which the femoral head H is fitted. An attachment panel 16 extends off of an upper portion of the medial shell 10 and has one or more openings 18, in this embodiment there are 5 openings 18. When the medial shell 10 is first put in place, two compression bone screws 60 are inserted to secure the shell 10 to the bone surface, likely in the second and forth openings 18.

The lateral shell 30 is designed in a similar manner to the medial shell 10, having an outer side 32 and an inner side 34. The inner side 32 is semi-circular and adapted to secure around one half of the femoral head H. An upper portion has a similar attachment panel 36 with four openings 38. A screw 70, such as an M5 machine screw, is inserted through one of the openings 38 and directly into the medial shell 10 to initially secure the two shells together. The other lateral shell openings 38 are configured to line up with medial shell openings 18 so that three additional compression bone screws may be inserted through both attachment panels 16, 36, and into the bone.

The liner 50 is a semi-spherical cup that is configured to fit on top of the femoral head H. In use, the liner 50, on top of the femoral head H, is inserted into the shell 10, 30, before the two shells are attached to one another.

A number of conventional materials may be used in the formation of the various components. For example, the modular neck 200 may be made from titanium. Similarly, each of the medial shell 10 and the lateral shell 30 may be made from titanium. The liner 50 is likely to be made of ultra-high molecular weight polyethylene. The femoral head H may be made of ceramic or cobalt chrome.

The disclosure herein has discussed the acetabular device and modular neck segment in terms of its use for temporary implantation during a hip distraction procedure to reposition the femur and allow for total hip arthroplasty (“THA”) to address chronic hip dislocation, however, it may also be suitable with other forms of distraction, for example, with a shoulder distraction.

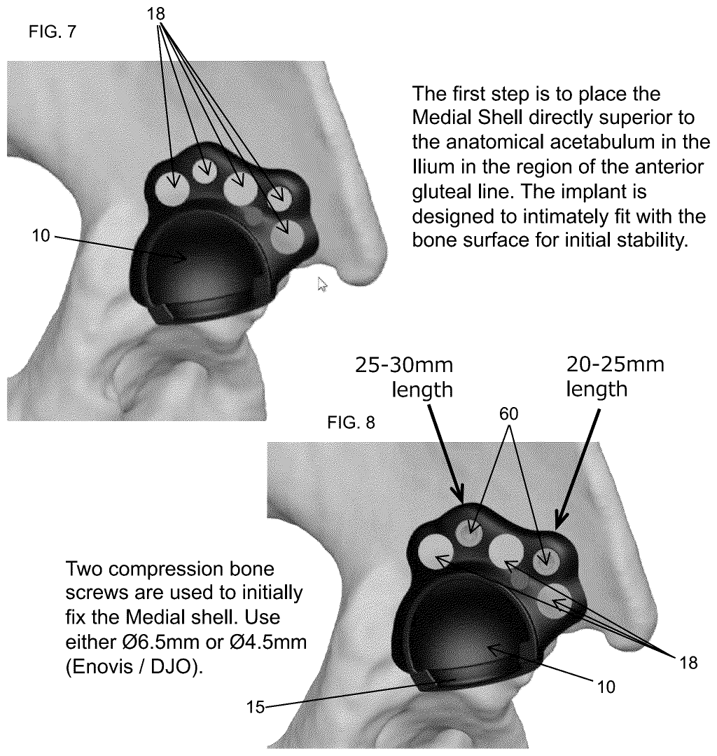

FIGS. 7-16 illustrate one method of using the device 100. In this example, the first step is to place the medial shell 10 directly superior to the anatomical acetabulum in the ilium in the region of the anterior gluteal line. The medial shell 10 is designed to intimately fit with the bone surface for initial stability. Once in the proper position, two compression bone screws are used to initially fix the medial shell 10 to the bone, likely using either Ø6.5 mm or Ø4.5 mm screws.

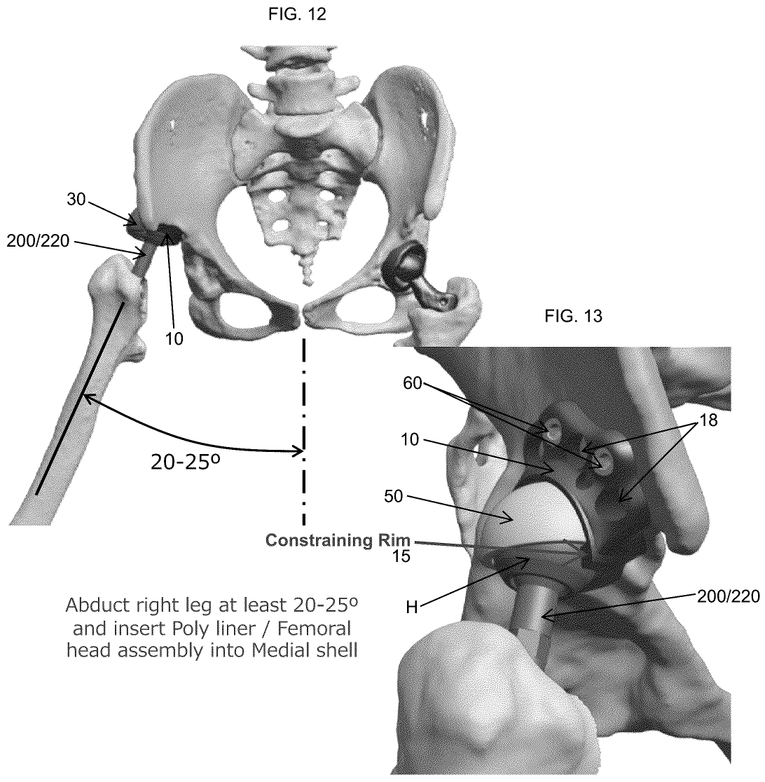

As noted, the taper neck segment of the modular neck 200 is designed to screw into the proximal end of a conventional IM hip nail N. With the medial shell 10 in place, the neck segment is attached to the IM nail and the femoral head of choice H is placed onto the taper of the neck segment, after which it is inserted into the medial shell 10 as shown in FIGS. 12 and 13. To make the insertion on a right leg, for example, abduct the right leg at least 20-25 degrees. The liner and femoral head fit snugly against the medial head 10 and above the constraining rim 15.

Next, the lateral shell 30 is placed over the line on the femoral head and the remaining bone screws are inserted through the lateral shell 30 and medial shell 10 to secure the entire assembly. In the embodiment shown in the example, there are two screws that only attach the medial shell to the bone and three that securely attach both the medial and lateral shell to the bone, with one screw that only attaches the lateral shell to the medial shell, however, it should be noted that the specific number of screws is only an example and it is possible to complete the assembly with more or fewer screws.

It is understood that the embodiments described herein are merely illustrative of the present invention. Variations in the construction of the acetabular device with a modular neck may be contemplated by one skilled in the art without limiting the intended scope of the invention herein disclosed.

What is claimed is:

1: A device adapted to secure a femoral head prosthesis, the device comprising:
a medial shell and a lateral shell, each of the medial shell and the lateral shell shaped to fit around a portion of the femoral head prosthesis;
the medial shell having a medial shell constraining rim on a lower portion of the medial shell, the lateral shell having a lateral shell constraining rim on a lower portion of the later shell, the medial shell constraining rim and lateral shell constraining rim configured to secure the medial shell and the lateral shell to a lower portion of the femoral head prosthesis, an upper portion of the femoral head prosthesis fitting against an inner side of the medial shell and an inner side of the lateral shell.

2: The device of claim 1, wherein the medial shell includes one or more medial shell openings that are configured to allow insertion of fasteners through the medial shell to attach the medial shell to a bone surface.

3: The device of claim 2, wherein the lateral shell includes one or more lateral shell openings that are configured to allow insertion of the fasteners through the lateral shell to attach the lateral shell to the medial shell.

4: The device of claim 3, wherein at least one of the medial shell openings and at least one of the later shell openings are configured to be in alignment such that a fastener passes through each of the lateral shell and the medial shell and into the bone surface.

5: The device of claim 4, further including a shell fastener that secures the lateral shell to the medial shell.

6: The device of claim 1, wherein a liner is placed over the femoral head prosthesis and into the medial shell and lateral shell.
